(19) United States
(12) Patent Application Publication
(10) Pub. No.: US 2024/0309565 A1
(43) Pub. Date: Sep. 19, 2024

(54) SHAPE MORPHING FABRICS AND ADAPTIVE FABRICS COMPRISING COMPRESSIBLE FIBERS

(71) Applicant: Sony Group Corporation, Tokyo (JP)

(72) Inventors: Silvia ROSSELLI, Stuttgart (DE); Elham KHODABAKHSHISHALAMZARI, Stuttgart (DE); Diana HENNIG, Stuttgart (DE); Anthony ROBERTS, Stuttgart (DE); Dennis CHERCKA, Stuttgart (DE); Vitor Angelo Fonseca DEICHMANN, Stuttgart (DE); Simon STAHL, Stuttgart (DE); Gabriele NELLES, Stuttgart (DE); Tomoaki TAKUMA, Stuttgart (DE); Simon HENNING, Stuttgart (DE); Akira ONO, Stuttgart (DE); Junko KATSURAKU, Stuttgart (DE); Ryosuke NAKAYAMA, Stuttgart (DE)

(73) Assignee: Sony Group Corporation, Tokyo (JP)

(21) Appl. No.: 18/283,453

(22) PCT Filed: Mar. 17, 2022
(86) PCT No.: PCT/EP2022/057067

§ 371 Date: Sep. 22, 2023

(30) Foreign Application Priority Data
Mar. 31, 2021 (EP) 21166281.2

(51) Int. Cl. D03D 15/50; A61F 7/00; A61F 13/08; D03D 15/283; D03D 15/292

(52) U.S. Cl. CPC D03D 15/50; A61F 7/007; A61F 13/08; D03D 15/283; D03D 15/292; A41B 2500/20; D10B 2331/02; D10B 2401/046; D10B 2501/021; D10B 2505/00; D10B 2509/02

(57) ABSTRACT

The field of the DISCLOSURE lies in adaptive materials for implementation in textiles, wearables and smart clothing. The present disclosure relates to functional fabrics or devices, comprising flexible fabrics or fiber-based materials which reversibly change shape upon stimulation with temperature and/or electrical (electrical field and/or charge) and/or upon applying tension. The present disclosure also relates to the use of said functional fabrics or devices, in particular for changing the hardness-softness in wearables or in parts of objects, or as variable friction layer in robotic grippers. The present disclosure also relates to wearable electronics, smart clothing, sport bandages and compression stockings, flexible sheet heaters. The present disclosure also relates to functional fabrics or devices and theirs uses in shape morphing fabrics, as soft/hard powered exoskeletons for posture corrective/supportive wearables or robotic artificial muscles.

### BACKGROUND

The field of the DISCLOSURE lies in adaptive materials for implementation in textiles, wearables and smart clothing.

The present disclosure relates to functional fabrics or devices, comprising flexible fabrics or fiber-based materials which reversibly change shape upon stimulation with temperature and/or electrical (electrical field and/or charge) and/or upon applying tension.

The present disclosure also relates to the use of said functional fabrics or devices, in particular for changing the hardness-softness in wearables or in parts of objects, or as variable friction layer in robotic grippers. The present disclosure also relates to wearable electronics, smart clothing, sport bandages and compression stockings, flexible sheet heaters.

The present disclosure also relates to functional fabrics or devices and theirs uses in shape morphing fabrics, as soft/hard powered exoskeletons for posture corrective/supportive wearables or robotic artificial muscles.

### DESCRIPTION OF THE RELATED ART

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present disclosure.

The research and applications of fiber materials are directly related to the daily life of the social populace and the development of relevant revolutionary manufacturing industries. However, conventional fibers and fiber products can no longer meet the requirements of automation and intellectualization in modern society, as well as people's consumption needs in their pursuit of smart, avant-garde, fashion and distinctiveness.

### SUMMARY

In the following, the elements of the invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine two or more of the explicitly described embodiments or which combine the one or more of the explicitly described embodiments with any number of the disclosed and/or preferred elements. Furthermore, any permutations and combinations of all described elements in this application should be considered disclosed by the description of the present application unless the context indicates otherwise.

The present disclosure provides a functional fabric or device comprising flexible fabrics or fiber-based materials which reversibly change shape upon stimulation with temperature and/or electrical (electrical field and/or charge) and/or upon applying tension.

The present disclosure provides the use of a fabric of the present disclosure for changing the hardness-softness in wearables.

The present disclosure provides the use of a fabric of the present disclosure for changing the hardness-softness in parts of objects or of whole objects for changing the comfortability and/or conformability of said objects.

The present disclosure provides the use of a fabric of the present disclosure as flexible sheet heater.

The present disclosure provides the use of a fabric of the present disclosure in textiles for control of pore size of said textile, in particular for selective permeability.

The present disclosure provides the use of a fabric of the present disclosure as sport bandage.

The present disclosure provides the use of a fabric of the present disclosure as compression stockings, which are suitable for customizable compression and comfortable fit.

The present disclosure provides the use of a fabric of the present disclosure as electronic hardware adapting its shape to the grasp of the user, and restoring its original smooth shape when not in use.

The present disclosure provides the use of a fabric of the present disclosure as adaptive fit wristband for gaming or other wearable.

The present disclosure provides the use of a fabric of the present disclosure as variable friction layer on robotic grippers.

The present disclosure provides wearable electronics or smart clothing, comprising a fabric according to the present disclosure.

The present disclosure provides a sport bandage, comprising a fabric according to the present disclosure.

The present disclosure provides compression stockings comprising a fabric according to the present disclosure, wherein the compression stockings are suitable for customizable compression and comfortable fit.

The present disclosure provides a flexible sheet heater, comprising a fabric of the present disclosure.

The present disclosure provides the use of a fabric or device of the present disclosure in textiles for control of pore size of said textile, in particular for selective permeability.

The present disclosure provides the use of a fabric or device of the present disclosure as shape morphing fabrics.

The present disclosure provides the use of a fabric or device of the present disclosure in shoes.

The present disclosure provides the use of a fabric or device of the present disclosure on wearables.

The present disclosure provides the use of a fabric or device of the present disclosure as soft/hard powered exoskeletons for posture corrective/supportive wearables.

The present disclosure provides the use of a fabric or device of the present disclosure as microfluidic actuators or circuits.

The present disclosure provides the use of a fabric or device of the present disclosure for changing the hardness-softness in parts of objects or of whole objects.

The present disclosure provides the use of a fabric or device of the present disclosure as sport bandage.

The present disclosure provides the use of a fabric or device of the present disclosure as compression stockings, which are suitable for customizable compression and comfortable fit.

The present disclosure provides the use of a fabric or device of the present disclosure as adaptive fit wristband for gaming or other wearable.

The present disclosure provides the use of a fabric or device of the present disclosure as robotic artificial muscles.

The present disclosure provides wearable electronics or smart clothing, comprising a fabric or device according to the present disclosure.

The present disclosure provides a shape morphing fabric, comprising a fabric or device according to the present disclosure.

The present disclosure provides a sport bandage, comprising a fabric or device according to the present disclosure.

The present disclosure provides compression stockings comprising a fabric or device according to the present disclosure, wherein the compression stockings are suitable for customizable compression and comfortable fit.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 shows extruded shape memory polymer (SMP) fibers,

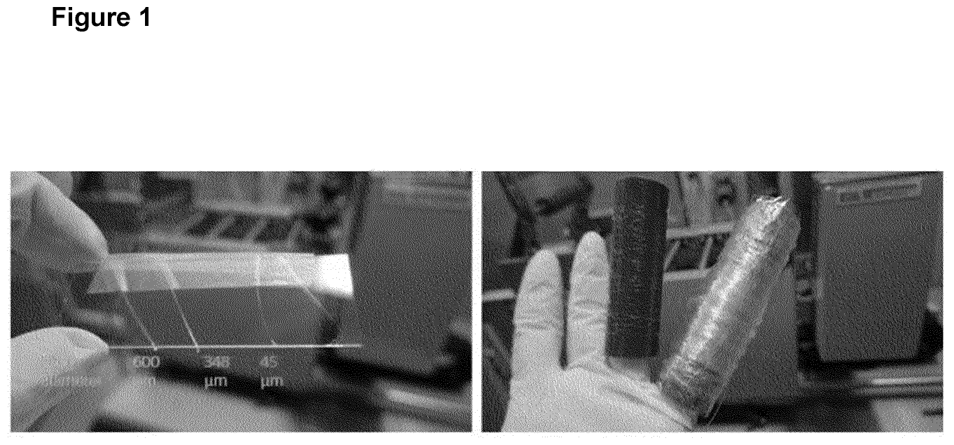

FIG. 2 shows the hard-soft property change of SMP films placed onto the surface of cold water (about 15° C.) and warm water (about 70° C.). A glass rod is pressed on both surfaces, showing rigid behavior of the cold film, whereas the warm film could be deformed and relaxed by impression.

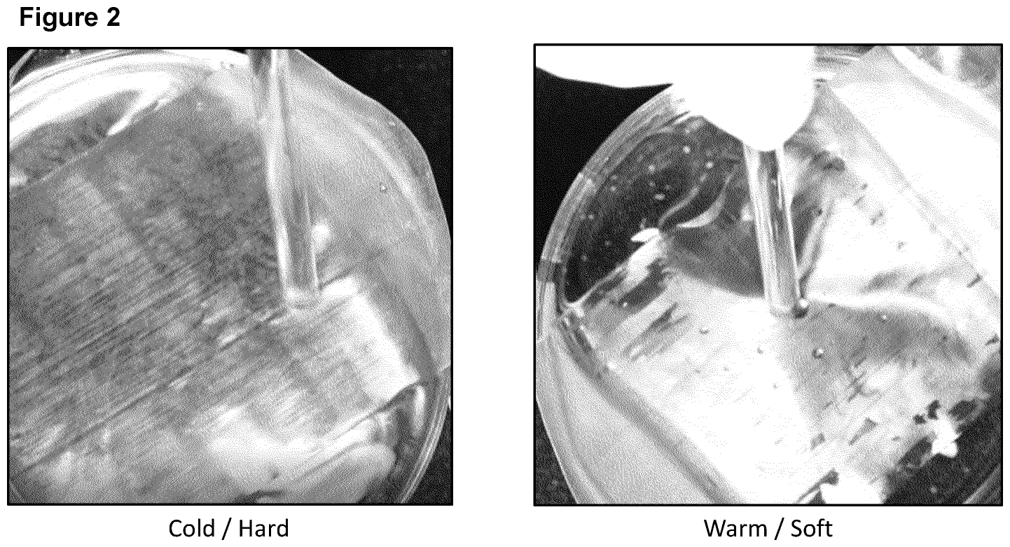

FIG. 3 shows the shape memory property. In warm water (about 70° C.), the SMP material is above its glass transition temperature, it is soft and can be bent. Bringing the deformed material into cold water (about 15° C.) froze its shape. Subsequent dipping into the warm water restored its initial shape.

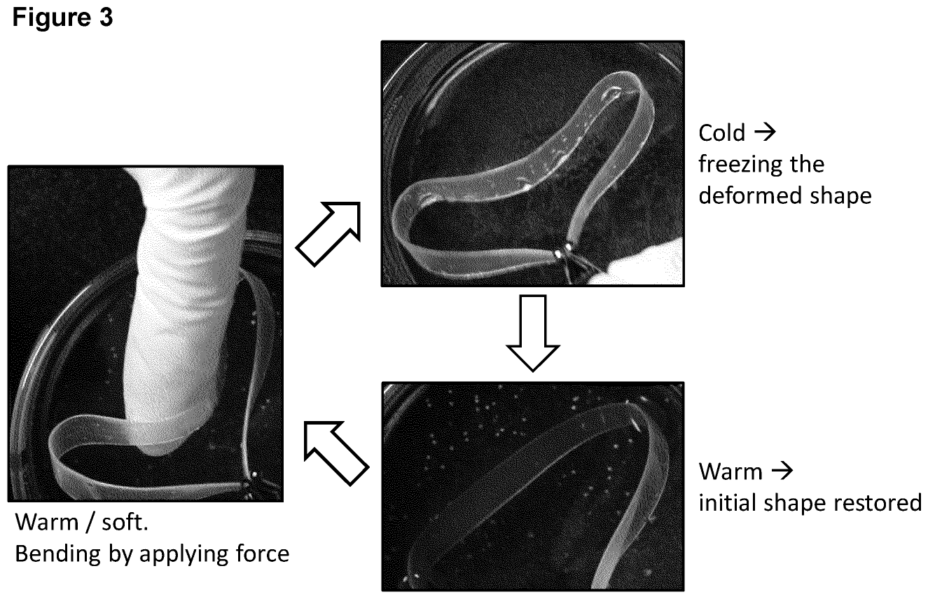

FIG. 4 shows a thin film heater, and the silver nanowire needles after heater fabrication.

FIG. 5 shows a SMP material stacked with a thin film heater that show flexibility at constant current.

FIG. 6 shows examples of single twisted synthetic yarn samples, single LLDPE fiber TSF (A) and Ag Nylon yarns (B).

FIG. 7 shows flexible and soft textile (top) and the shaped textile after applying heat (bottom).

### DETAILED DESCRIPTION OF THE EMBODIMENTS

As discussed above, the present disclosure provides a functional fabric or device. Said functional fabric or device comprises flexible fabrics or fiber-based materials which reversibly change shape upon stimulation with temperature and/or electrical (electrical field and/or charge) and/or upon applying tension.

Preferably, said fabric or device has different states it reversibly changes in between in dependence on said external stimulus, such as two states.

In one embodiment, the fabric or device has a soft and flexible state and a hard and less flexible, or even rigid, state (soft-hard).

In one embodiment, the fabric or device has a state where it has more grip and a state where it has less grip/smooth state.

In one embodiment, the fabric or device has a contracted state and an expanded state.

In a preferred embodiment, the fabric of the present disclosure comprises said flexible fabric or fiber-based material which comprises
(i) fibers of shape memory material(s), preferably shape memory polymer(s) (SMP), or
(ii) fibers coated or laminated with shape memory material(s), preferably shape memory polymer(s) (SMP),
wherein said fibers (i) or (ii) are woven or knitted forming a pattern, or
(iii) films of shape memory material(s), preferably shape memory polymer(s) (SMP),
or
(iv) structures of shape memory materials (e.g. patterned), which are preferably fabricated by fused deposition molding such as 3D printing.

Preferably, the SMP is an olefin-based polymer or a polyurethane-based polymer.

The SMP preferably has a glass transition temperature (Tg) in range from about 15° C. to 55° C., more preferably 20° C. to 45° C., such as about 30° C. or about 45° C.

Said SMP is extrusion molded into said fiber or film.

Examples of SMPs are an a-Olefin Copolymer or ether-type polyurethane polymer.

Thermoplastic SMPs having various Tg are also suitable, which are optionally compounded among each other by extrusion molding.

The patterning for said materials (iv) can be different types of SMP, and/or different types of materials in general, such as combined with an elastomer material.

Preferably, the fabric changes its shape induced by external mechanical deformation at warm conditions, which are above the Tg. Said changed shape can be kept during cooling (“frozen”) and retains after said cooling below Tg. The initial shape (memory shape) can be restored in warm conditions/by another heating above Tg, as long as there is no significant external force applied.

Preferably, keeping the fabric warm or cold gives control over its stiffness state, thereby the fabric or device changes between glassy (hard feel) and rubbery (soft feel) state when altering the temperature.

In one embodiment, the film of shape memory material(s), preferably shape memory polymer(s) (SMP), is coated with conductive material(s), wherein furthermore electrodes are applied to.

Preferably, said conductive material(s) is/are flexible and optionally also stretchable, i.e. elastically or plastically deformable. Examples for conductive materials are metal/conductive paste or metallic nanowire assemblies, such as silver-based or carbon-based elastomer paste or silver nanowire assemblies.

In such embodiment, the stiffness state of the fabric is electrically controllable via applying a voltage across/a current through the layer of conductive material(s) (i.e. through the Joule heating layer), causing Joule heating, thereby heating up the fabric above its Tg as well and thereby lowering its stiffness.

In one embodiment, the fabric or device further comprises means for heat insulation, such as a heat insulating (possibly elastomeric) textile, film or foam

As discussed above, the present disclosure provides the use of a fabric of the present disclosure for changing the hardness-softness in wearables.

Examples are shoes and clothing, e.g. socks, gloves, shirts, belts, skirts, kilts, trousers, scarfs, shawls, onesies, wigs, or hats.

As discussed above, the present disclosure provides the use of a fabric of the present disclosure for changing the hardness-softness in parts of objects or of whole objects for changing the comfortability and/or conformability of said objects.

Examples are seats and mattresses.

As discussed above, the present disclosure provides the use of a fabric of the present disclosure as flexible sheet heater.

As discussed above, the present disclosure provides the use of a fabric of the present disclosure in textiles for control of pore size of said textile, in particular for selective permeability.

As discussed above, the present disclosure provides the use of a fabric of the present disclosure as sport bandage.

As discussed above, the present disclosure provides the use of a fabric of the present disclosure as compression stockings, which are suitable for customizable compression and comfortable fit, e.g. for travel and sports.

The present disclosure provides the use of a fabric of the present disclosure as electronic hardware adapting its shape to the grasp of the user, and restoring its original smooth shape when not in use.

As discussed above, the present disclosure provides the use of a fabric of the present disclosure as adaptive fit wristband for gaming or other wearable, which can preferably transmit haptic signals.

As discussed above, the present disclosure provides the use of a fabric of the present disclosure as variable friction layer on robotic grippers.

As discussed above, the present disclosure provides wearable electronics or smart clothing, comprising a fabric according to the present disclosure.

As discussed above, the present disclosure provides a sport bandage, comprising a fabric according to the present disclosure. The fabric or device is optionally furthermore breathable and supports cooling and/or adapts to shape of swelling. The sport bandage preferably adapts during healing.

As discussed above, the present disclosure provides compression stockings comprising a fabric according the present disclosure.

Said compression stockings are suitable for customizable compression and comfortable fit, such as for travel and sports.

As discussed above, the present disclosure provides a flexible sheet heater, comprising a fabric of the present disclosure.

In a preferred embodiment, a fabric or device of the present disclosure comprises said flexible fabric or fiber-based material which comprises
(i) twisted synthetic fibers (TSFs), preferably twisted polymer fibers,
or
(ii) actuator fibers, such as TSFs, coated, laminated or coiled with conductive material(s).

Said fibers (i) or (ii) are woven or knitted with other fibers, preferably stretchable (textile) fibers, optionally forming a pattern.

Preferably, the TSFs comprise
single or multiple polymer fibers, such as polyethylene, polyamide, or
carbon nanotube yarns.

Preferably, the polyethylene fibers are linear low density polyethylene (LLDPE). They can also be high density polyethylene (HDPE).

Preferably, the polyamide fibers are nylon, such as nylon 6 monofilament or yarn, nylon 6.6 monofilament or yarn.

Thereby, the TSF exhibit one or more of the following characteristics: a low fiber density, a low specific heat and a low spring index (i.e. the ratio of coil and fiber diameter).

Preferably, the TSF is formed by twisting and coiling of a fiber with an anisotropy of the thermal expansion coefficient.

This implies that the expansion in fiber direction is negative, whereas it is positive perpendicular to it. The large anisotropic thermal strain of this type of drawn crystalline polymer originates from stretched tie-molecules, causing large entropy elasticity along the drawn axis, and resulting in negative linear thermal expansion of the fiber.

In one embodiment, the conductive material(s) the actuator fibers are coated, laminated or coiled with, are metals, metallic or conductive polymers, e.g. silver, steel, copper or carbon, or poly(3,4-ethylenedioxythiophene) polystyrene sulfonate (PEDOT:PSS) or composites thereof.

Preferably, the TSFs contract during heating, such as Joule heating or an increase in environmental temperature. Optionally the fabric further comprises fibers capable of resistive heating.

In one embodiment, the fabric or device further comprises means for passive and/or active cooling, such as an air fan, a cooling water circuit or a Peltier element.

In one embodiment, the fabric or device further comprises means for heat insulation, such as a heat insulating (possibly elastomeric) textile, film or foam

The present disclosure provides the use of a fabric or device of the present disclosure as shape morphing fabrics.

Said use is preferably for use in posture control.

The present disclosure provides the use of a fabric or device of the present disclosure as shape morphing fabrics.

For example, as components in gaming controllers, headphones, furniture.

The present disclosure provides the use of a fabric or device of the present disclosure in shoes, such as in shoe soles.

The present disclosure provides the use of a fabric or device of the present disclosure on wearables. For example, as gaming gloves.

The present disclosure provides the use of a fabric or device of the present disclosure as soft/hard powered exoskeletons for posture corrective/supportive wearables.

The present disclosure provides the use of a fabric or device of the present disclosure as microfluidic actuators or circuits.

The present disclosure provides the use of a fabric or device of the present disclosure for changing the hardness-softness in parts of objects or of whole objects.

For example, mobile phone cases.

The present disclosure provides the use of a fabric or device of the present disclosure as sport bandage.

The present disclosure provides the use of a fabric or device of the present disclosure as compression stockings, which are suitable for customizable compression and comfortable fit, such as for travel and sports.

The present disclosure provides the use of a fabric or device of the present disclosure as adaptive fit wristband for gaming or other wearable.

The present disclosure provides the use of a fabric or device of the present disclosure as robotic artificial muscles.

The present disclosure provides wearable electronics or smart clothing, comprising a fabric or device according to the present disclosure.

The present disclosure provides a shape morphing fabric, comprising a fabric or device according to the present disclosure.

The present disclosure provides a sport bandage, comprising a fabric or device according to the present disclosure. The fabric or device is optionally furthermore breathable and supports cooling and/or adapts to shape of swelling. The sport bandage preferably adapts during healing.

The present disclosure provides compression stockings comprising a fabric or device according to the present disclosure, wherein the compression stockings are suitable for customizable compression and comfortable fit, such as for travel and sports.

Note that the present technology can also be configured as described below.

(1) A functional fabric or device, comprising flexible fabrics or fiber-based materials which reversibly change shape upon stimulation with temperature and/or electrical (electrical field and/or charge) and/or upon applying tension,
wherein preferably said fabric has different states it reversibly changes in between in dependence on said stimulation, such as two states, e.g.
a soft and flexible state and a hard and less flexible state (soft-hard), and/or
a state where it has more grip and a state where it has less grip/smooth state, and/or
a contracted state and an expanded state.
   (2) A fabric of embodiment (1), wherein said flexible fabric or fiber-based material comprises
(i) fibers of shape memory material(s), preferably shape memory polymer(s) (SMP), or
(ii) fibers coated or laminated with shape memory material(s), preferably shape memory polymer(s) (SMP),
wherein said fibers (i) or (ii) are woven or knitted forming a pattern,
or
(iii) films of shape memory material(s), preferably shape memory polymer(s) (SMP),
or
(iv) structures of shape memory materials (e.g. patterned), which are preferably fabricated by fused deposition molding, such as 3D printing.
   (3) The fabric of embodiment (1) or (2), wherein the SMP is an olefin-based or polyurethane-based polymer, preferably having a glass transition temperature (Tg) in range from about 15° C. to 55° C., more preferably 20° C. to 45° C., such as about 30° C. or about 45° C.,
which is extrusion molded into said fiber or film,
or wherein thermoplastic SMPs having various Tg are used, which are optionally compounded among each other by extrusion molding.
   (4) The fabric of any one of embodiments (1) to (3), wherein the fabric changes its shape induced by external mechanical deformation at warm conditions, above Tg,
wherein said changed shape is kept during cooling and retains after said cooling below Tg, and
wherein the initial shape (memory shape) is restored by another heating above Tg, as long as there is no significant external force applied.
   (5) The fabric of any of the preceding embodiments, wherein keeping the fabric warm or cold gives control over its stiffness state, thereby the fabric or device changes between rubbery (soft feel) and glassy (hard feel) state when altering the temperature.
   (6) The fabric of any of the preceding embodiments, wherein the film of shape memory material(s), preferably shape memory polymer(s) (SMP), is coated with conductive material(s), wherein furthermore electrodes are applied to,
wherein said conductive material(s) is/are flexible and optionally also stretchable, i.e. elastically or plastically deformable, such as metal/conductive paste or metallic nanowire assemblies, e.g. silver-based or carbon-based elastomer paste, or silver nanowire assemblies,
wherein the stiffness state of the fabric is electrically controllable
   via applying a voltage across/current through the layer of conductive material(s), causing Joule heating,
   thereby heating up the fabric above its Tg as well and thereby lowering its stiffness.
      (7) The fabric or device of any of the preceding embodiments, wherein the fabric or device further comprises means for heat insulation, such as a heat insulating (possibly elastomeric) textile, film or foam
      (8) Use of a fabric of any one of embodiments (1) to (7),
for changing the hardness-softness in wearables, such as shoes, and clothing (e.g. socks, gloves, shirts, belts, skirts, kilts, trousers, scarfs, shawls, onesies, wigs, hats),
for changing the hardness-softness in parts of objects or of whole objects for changing the comfortability and/or conformability of said objects, such as seats, or mattresses,
as flexible sheet heater,
in textiles for control of pore size of said textile, in particular for selective permeability,
as sport bandage,
as compression stockings, which are suitable for customizable compression and comfortable fit, e.g. for travel and sports,
as electronic hardware adapting its shape to the grasp of the user, and restoring its original smooth shape when not in use,
as adaptive fit wristband for gaming or other wearable, which can preferably transmit haptic signals, and/or
as variable friction layer on robotic grippers.
   (9) Wearable electronics or smart clothing, comprising a fabric according to any one of embodiments (1) to (7).
   (10) A sport bandage, comprising
a fabric according to any one of embodiments (1) to (7),
wherein the fabric or device optionally is furthermore breathable and supports cooling and/or adapts to shape of swelling, wherein the sport bandage preferably adapts during healing.
   (11) Compression stockings comprising
a fabric according to any one of embodiments (1) to (7),
   wherein the compression stockings are suitable for customizable compression and comfortable fit, e.g. for travel and sports.
   (12) A flexible sheet heater, comprising a fabric of embodiment (7).
   (13) A fabric or device of embodiment (1), wherein said flexible fabric or fiber-based material comprises
(i) twisted synthetic fibers (TSFs), preferably twisted polymer fibers, or
(ii) actuator fibers, such as TSFs, coated, laminated or coiled with conductive material(s),
wherein said fibers (i) or (ii) are woven or knitted with other fibers, preferably stretchable (textile) fibers, optionally forming a pattern.
   (14) The fabric or device of embodiment (13), wherein the TSFs comprise
single or multiple polymer fibers, such as
   polyethylene, preferably linear low density polyethylene (LLDPE) or high density polyethylene (HDPE),
   polyamide, preferably nylon, such as nylon 6 monofilament or yarn, nylon 6.6 monofilament or yarn,
or carbon nanotube yarns,
   and wherein the TSF exhibit one or more of the following characteristics: a low fiber density, a low specific heat and a low spring index (i.e. the ratio of coil and fiber diameter).
   (15) The fabric or device of embodiment (13) or (14), wherein the TSF is formed by twisting and coiling of a fiber with an anisotropy of the thermal expansion coefficient.
   (16) The fabric or device of any one of embodiments (13) to (15), wherein the conductive material(s) the actuator fibers are coated, laminated or coiled with, are metals, metallic or conductive polymers, e.g. silver, steel, copper or carbon, or PEDOT:PSS or composites thereof.
   (17) The fabric or device of any one of embodiments (13) to (16), wherein the TSFs contract during heating, such as Joule heating or increase in environmental temperature, and wherein optionally the fabric further comprises fibers capable of resistive heating.
   (18) The fabric or device of any one of embodiments (13) to (17), wherein the fabric or device further comprises means for passive and/or active cooling, such as an air fan, a cooling water circuit or a Peltier element.
   (19) The fabric or device of any one of embodiments (13) to (17), wherein the fabric or device further comprises means for heat insulation, such as a heat insulating (possibly elastomeric) textile, film or foam
   (20) Use of a fabric or device of any one of embodiments (13) to (19),
in textiles for control of pore size of said textile, in particular for selective permeability,
as shape morphing fabrics, preferably for use in posture control,
as shape morphing fabrics, e.g. as components in gaming controllers, headphones, furniture,
in shoes, such as shoe soles,
on wearables, such as gaming gloves,
as soft/hard powered exoskeletons for posture corrective/supportive wearables,
as microfluidic actuators or circuits,
for changing the hardness-softness in parts of objects or of whole objects, such as mobile phone cases,
as sport bandage,
as compression stockings, which are suitable for customizable compression and comfortable fit, e.g. for travel and sports
as adaptive fit wristband for gaming or other wearable, which can preferably transmit haptic signals, and/or
as robotic artificial muscles.
   (21) Wearable electronics or smart clothing, comprising a fabric or device according to any one of embodiments (13) to (19).
   (22) A shape morphing fabric, comprising
a fabric or device according to any one of embodiments (13) to (19).
   (23) A sport bandage, comprising
a fabric or device according to any one of embodiments (13) to (19),
wherein the fabric or device optionally is furthermore breathable and supports cooling and/or adapts to shape of swelling,
   wherein the sport bandage preferably adapts during healing.
   (24) Compression stockings comprising
a fabric or device according to any one of embodiments (13) to (19), wherein the compression stockings are suitable for customizable compression and comfortable fit, e.g. for travel and sports.

The advanced fiber-shaped and fiber-form electronics with most desired designability and integration features have been explored and developed by the present inventors. Advanced fiber-based products, such as wearable electronics and smart clothing, can be employed as a second skin to enhance information exchange between humans and the external environment. Active and reactive (adaptive) materials with respect to the environmental and/or the personal situations will increase customer value, opening new possibilities for personalization, emotion expression and product utilization.

The major object of this disclosure is to provide flexible fiber-shaped multifunctional devices, including fiber-based materials having different functionalities and being adaptive/stimuli responsive, meaning changing one or more material properties depending from the stimulus applied.

### EXAMPLES

### Example 1: Shape Memory Polymer Film with Flexible Sheet Heater

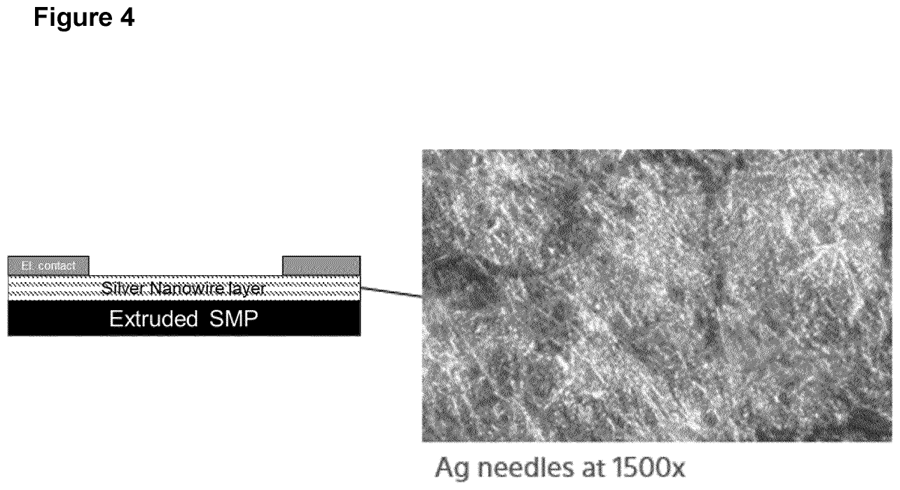

As outlined in FIG. 4, a conductive layer of silver nanowires was deposited on top of an extruded alpha-olefin shape memory polymer (SMP) film. Copper tape electrodes were bonded to the silver nanowires layer. The needle-like structure of the conductive layer was confirmed by optical microscopy.

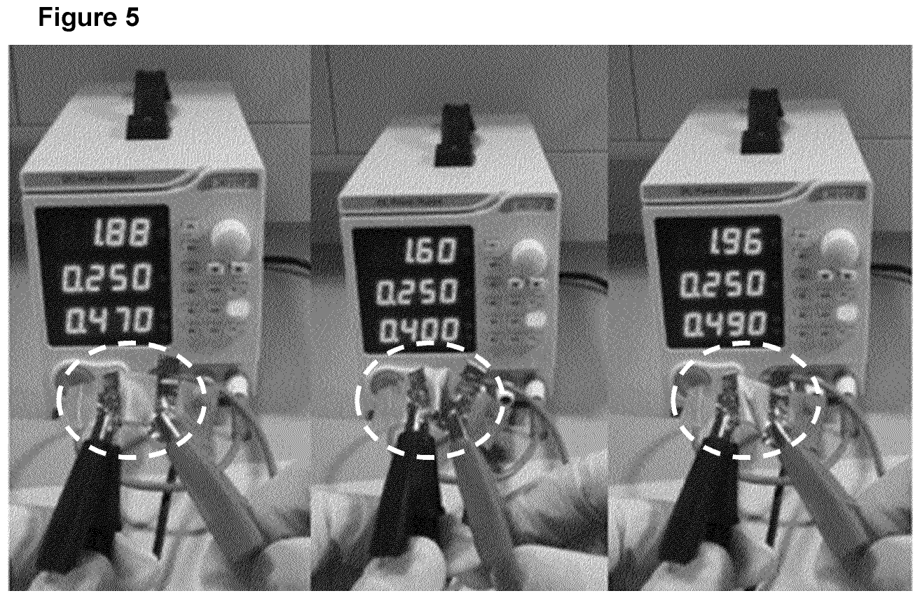

The softening and hardening behavior of the stack was confirmed by connection to a DC voltage source with a limited current of 0.25 A. When electrically connected and heated up, the SMP softened and voltages of up to 2V were observed, then after disconnection from the electrical power supply, the SMP gradually hardened as it cooled down to room temperature (see FIG. 5).

### Example 2: Twisted Synthetic Fibers

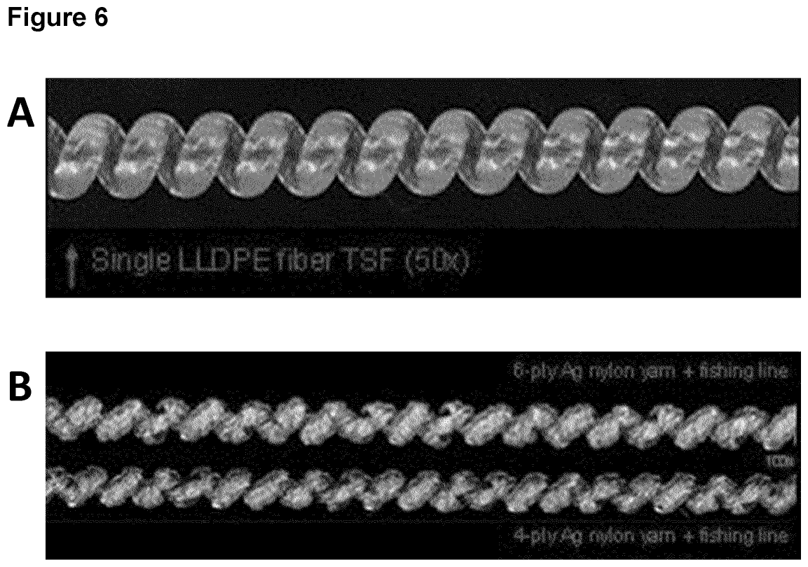

One or two silver Nylon 6,6 4-ply polymer fibers were twisted and coiled, and subsequently mounted in a frame under tension to prevent uncoiling. After annealing at 180° C. for one hour the Twisted Synthetic Fibers (TSFs) were prepared. Microscope pictures of the resulting TSFs are shown in FIG. 6.

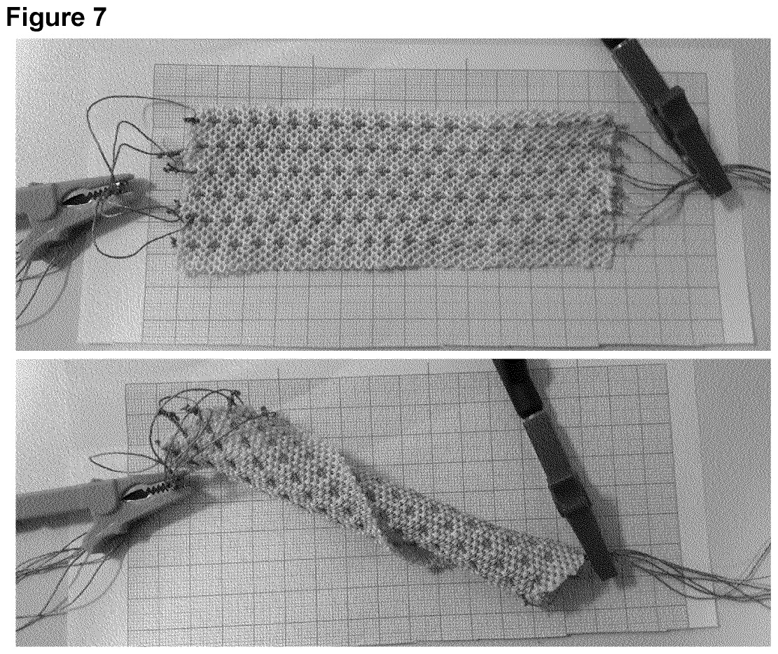

TSFs obtained with the above procedure were woven in parallel through a fabric. After connecting to 7 V DC voltage (constant 3 A) in parallel, contraction of the yarn resulted in a roll-up actuation of the fabric, see FIG. 7.

The features of the present disclosure disclosed in the specification, the claims, and/or in the accompanying figures may, both separately and in any combination thereof, be material for realizing the disclosure in various forms thereof. Thus, the foregoing discussion discloses and describes merely exemplary embodiments of the present disclosure. As will be understood by those skilled in the art, the present disclosure may be embodied in other specific forms without departing from the spirit or essential characteristics thereof. Accordingly, the disclosure of the present disclosure is intended to be illustrative, but not limiting of the scope of the disclosure, as well as other claims. The disclosure, including any readily discernible variants of the teachings herein, define, in part, the scope of the foregoing claim terminology such that no inventive subject matter is dedicated to the public.

1. A functional fabric or device, comprising flexible fabrics or fiber-based materials which reversibly change shape upon stimulation with temperature and/or electrical (electrical field and/or charge) and/or upon applying tension,
wherein preferably said fabric has different states it reversibly changes in between in dependence on said stimulation, such as two states, e.g.
a soft and flexible state and a hard and less flexible state (soft-hard), and/or
a state where it has more grip and a state where it has less grip/smooth state, and/or
a contracted state and an expanded state.

2. The fabric of claim 1, wherein said flexible fabric or fiber-based material comprises:
(i) fibers of shape memory material(s), preferably shape memory polymer(s) (SMP), or
(ii) fibers coated or laminated with shape memory material(s), preferably shape memory polymer(s) (SMP),
wherein said fibers (i) or (ii) are woven or knitted forming a pattern,
or
(iii) films of shape memory material(s), preferably shape memory polymer(s) (SMP),
or
(iv) structures of shape memory materials (e.g. patterned), which are preferably fabricated by fused deposition molding, such as 3D printing.

3. The fabric of claim 2, wherein the SMP is an olefin-based or polyurethane-based polymer, preferably having a glass transition temperature (Tg) in range from about 15° C. to 55° C., more preferably 20° C. to 45° C., such as about 30° C. or about 45° C.,
which is extrusion molded into said fiber or film,
or wherein thermoplastic SMPs having various Tg are used, which are optionally compounded among each other by extrusion molding.

4. The fabric of claim 3, wherein the fabric changes its shape induced by external mechanical deformation at warm conditions, above Tg, wherein said changed shape is kept during cooling and retains after said cooling below Tg, and wherein the initial shape (memory shape) is restored by another heating above Tg, as long as there is no significant external force applied.

5. The fabric of claim 4, wherein keeping the fabric warm or cold gives control over its stiffness state, thereby the fabric or device changes between rubbery (soft feel) and glassy (hard feel) state when altering the temperature.

6. The fabric of claim 2, wherein the film of shape memory material(s), preferably shape memory polymer(s) (SMP), is coated with conductive material(s), wherein furthermore electrodes are applied to,
wherein said conductive material(s) is/are flexible and optionally also stretchable (i.e. elastically or plastically deformable), such as metal/conductive paste or metallic nanowire assemblies, e.g. silver-based or carbon-based elastomer paste, or silver nanowire assemblies,
wherein the stiffness state of the fabric is electrically controllable
via applying a voltage across/current through the layer of conductive material(s), causing Joule heating,
thereby heating up the fabric above its Tg as well and thereby lowering its stiffness.

7. The fabric or device of claim 1, wherein the fabric or device further comprises means for heat insulation, such as a heat insulating (possibly elastomeric) textile, film or foam

8. Use of a fabric of claim 1,
for changing the hardness-softness in wearables, such as shoes, and clothing (e.g. socks, gloves, shirts, belts, skirts, kilts, trousers, scarfs, shawls, onesies, wigs, hats),
for changing the hardness-softness in parts of objects or of whole objects for changing the comfortability and/or conformability of said objects, such as seats, or mattresses,
as flexible sheet heater,
in textiles for control of pore size of said textile, in particular for selective permeability,
as sport bandage,
as compression stockings, which are suitable for customizable compression and comfortable fit, e.g. for travel and sports,
as electronic hardware adapting its shape to the grasp of the user, and restoring its original smooth shape when not in use,
as adaptive fit wristband for gaming or other wearable, which can preferably transmit haptic signals, and/or
as variable friction layer on robotic grippers.

9. Wearable electronics or smart clothing, comprising a fabric according to claim 1.

10. A sport bandage, comprising
a fabric according to claim 1,
wherein the fabric or device optionally is furthermore breathable and supports cooling and/or adapts to shape of swelling,
wherein the sport bandage preferably adapts during healing.

11. Compression stockings comprising
a fabric according to claim 1,
wherein the compression stockings are suitable for customizable compression and comfortable fit, e.g. for travel and sports.

12. A flexible sheet heater, comprising a fabric of claim 6.

13. A fabric or device of claim 1, wherein said flexible fabric or fiber-based material comprises
(i) twisted synthetic fibers (TSFs), preferably twisted polymer fibers,
or
(ii) actuator fibers, such as TSFs coated, laminated or coiled with conductive material(s),
wherein said fibers (i) or (ii) are woven or knitted with other fibers, preferably stretchable (textile) fibers, optionally forming a pattern.

14. The fabric or device of claim 13, wherein the TSFs comprise
single or multiple polymer fibers, such as
polyethylene, preferably linear low density polyethylene (LLDPE) or high density polyethylene (HDPE),
polyamide, preferably nylon, such as nylon 6 monofilament or yarn, nylon 6.6 monofilament or yarn,
or carbon nanotube yarns,
and wherein the TSF exhibit one or more of the following characteristics: a low fiber density, a low specific heat and a low spring index (i.e. the ratio of coil and fiber diameter).

15. The fabric or device of claim 13, wherein the TSF is formed by twisting and coiling of a fiber with an anisotropy of the thermal expansion coefficient.

16. The fabric or device of claim 13, wherein the conductive material(s) the actuator fibers are coated, laminated or coiled with, are metals, metallic or conductive polymers, e.g. silver, steel, copper or carbon, or PEDOT:PSS or composites thereof.

17. The fabric or device of claim 13,
wherein the TSFs contract during heating, such as Joule heating or increase in environmental temperature,
and wherein optionally the fabric further comprises fibers capable of resistive heating.

18. The fabric or device of claim 13, wherein the fabric or device further comprises means for passive and/or active cooling, such as an air fan, a cooling water circuit or a Peltier element.

19. The fabric or device of claim 13, wherein the fabric or device further comprises means for heat insulation, such as a heat insulating (possibly elastomeric) textile, film or foam

20. Use of a fabric or device of claim 13,
in textiles for control of pore size of said textile, in particular for selective permeability,
as shape morphing fabrics, preferably for use in posture control,
as shape morphing fabrics, e.g. as components in gaming controllers, headphones, furniture,
in shoes, such as shoe soles,
on wearables, such as gaming gloves,
as soft/hard powered exoskeletons for posture corrective/supportive wearables,
as microfluidic actuators or circuits,
for changing the hardness-softness in parts of objects or of whole objects, such as mobile phone cases,
as sport bandage,
as compression stockings, which are suitable for customizable compression and comfortable fit, e.g. for travel and sports
as adaptive fit wristband for gaming or other wearable, which can preferably transmit haptic signals, and/or
as robotic artificial muscles.

21. Wearable electronics or smart clothing, comprising a fabric or device according to claim 13.

22. A shape morphing fabric, comprising
a fabric or device according to claim 13.

23. A sport bandage, comprising
a fabric or device according to claim 13,
wherein the fabric or device optionally is furthermore breathable and supports cooling and/or adapts to shape of swelling,
wherein the sport bandage preferably adapts during healing.

24. Compression stockings comprising
a fabric or device according to claim 13,
wherein the compression stockings are suitable for customizable compression and comfortable fit, e.g. for travel and sports.
